(19) United States
(12) Patent Application Publication
(10) Pub. No.: US 2012/0245666 A1
(43) Pub. Date: Sep. 27, 2012

(54) Implantable Auditory Prosthesis with Temporary Connector

(75) Inventors: Claude Jolly, Innsbruck (AT); Thomas Lenarz, Hannover (DE)

(73) Assignee: MED-EL ELEKTROMEDIZINISCHE GERAETE GMBH, Innsbruck (AT)

(21) Appl. No.: 13/422,113

(22) Filed: Mar. 16, 2012

Related U.S. Application Data
(60) Provisional application No. 61/467,472, filed on Mar. 25, 2011.

(51) Int. Cl. A61N 1/05

(52) U.S. Cl. 607/137

(57) ABSTRACT

An implantable electrode arrangement for an ear implant system is described. A proximal electrode lead has electrode wires for carrying one or more electrical stimulation signals. A distal electrode array has electrode contacts each forming a terminal end of an electrode wire for applying the electrical stimulation signals to target neural tissue, and one or more intra-operative sensors for generating insertion sensing signals during surgical insertion of the electrode array into the target tissue. An intra-operative electrode section has a sensor connector for providing a temporary connection of one or more external measurement arrangements to the one or more intra-operative sensors during the surgical insertion of the electrode array without being functional after the surgical insertion.

This application claims priority from U.S. Provisional Patent 61/467,472, filed Mar. 25, 2011, which is incorporated herein by reference.

### TECHNICAL FIELD

The present invention relates to medical implants, and more specifically to ear implant systems such as cochlear implants, auditory brainstem implants, and vestibular implants.

### BACKGROUND ART

A normal ear transmits sounds as shown in FIG. 1 through the outer ear 101 to the tympanic membrane (eardrum) 102, which moves the bones of the middle ear 103, which in turn vibrate the oval window and round window openings of the cochlea 104. The cochlea 104 is a long narrow duct wound spirally about its axis for approximately two and a half turns. The cochlea 104 includes an upper channel known as the scala vestibuli and a lower channel known as the scala tympani, which are connected by the cochlear duct. The scala tympani forms an upright spiraling cone with a center called the modiolar where the spiral ganglion cells of the acoustic nerve 113 reside. In response to received sounds transmitted by the middle ear 103, the fluid filled cochlea 104 functions as a transducer to generate electric pulses that are transmitted to the cochlear nerve 113, and ultimately to the brain. Hearing is impaired when there are problems in the ability to transduce external sounds into meaningful action potentials along the neural substrate of the cochlea 104.

In some cases, hearing impairment can be addressed by an auditory prosthesis system such as a cochlear implant that electrically stimulates auditory nerve tissue with small currents delivered by multiple electrode contacts distributed along an implant electrode. FIG. 1 shows some components of a typical cochlear implant system where an external microphone provides an audio signal input to an external signal processing stage 111 which implements one of various known signal processing schemes. The processed signal is converted by the external signal processing stage 111 into a digital data format, such as a sequence of data frames, for transmission into a receiver processor in an implant housing 108. Besides extracting the audio information, the receiver processor in the implant housing 108 may perform additional signal processing such as error correction, pulse formation, etc., and produces a stimulation pattern (based on the extracted audio information) that is sent through an electrode lead 109 to an implanted electrode array 112 which penetrates into the cochlea 104 through a surgical opening called a cochleostomy. Typically, this electrode array 112 includes multiple electrode contacts 110 on its surface that deliver the stimulation signals to adjacent neural tissue of the cochlea 104 which the brain of the patient interprets as sound. The individual electrode contacts 110 may be activated sequentially, or simultaneously in one or more contact groups.

In some patients, the cochlea 104 itself is damaged so that a cochlear implant system as such is not a treatment option. One alternative for some such patients is an Auditory Brain Stem Implant (ABI) system which is somewhat like a cochlear implant, but which instead implants a stimulation electrode into auditory tissue in the brainstem of the patient.

Insertion and placement and insertion of the electrode array 112 into the cochlea 104 (or the auditory brainstem tissue in the case of an ABI electrode) causes trauma to the target tissue due to the rigidity, friction, and impact of moving the device through the cochlea 104. For example, insertion of the electrode array 112 may damage soft tissues, membranes, thin bony shelves, blood vessels, neural elements, etc. In the case of multiple insertions, the damage can accumulate. Thus, designers of the electrode array 110 work hard to ensure that it is soft and flexible to minimize the insertion trauma.

### SUMMARY

Embodiments of the present invention are directed to an implantable electrode arrangement for an ear implant system. A proximal electrode lead has electrode wires for carrying one or more electrical stimulation signals. A distal electrode array has electrode contacts each forming a terminal end of an electrode wire for applying the electrical stimulation signals to target neural tissue, and one or more intra-operative sensors for generating insertion sensing signals during surgical insertion of the electrode array into the target tissue. An intra-operative electrode section has a sensor connector for providing a temporary connection of one or more external measurement arrangements to the one or more intra-operative sensors during the surgical insertion of the electrode array without being functional after the surgical insertion.

In further specific embodiments, the insertion signals include dynamic real time sensing signals. The intra-operative sensors may include optical sensors, inductive sensors, and chemical sensors, and at least one of the intra-operative sensors may be separate from the electrode contacts. There also may be a connector cap for covering the sensor connector following the surgical insertion of the electrode array into the target tissue. The connector cap may be adapted to electrically isolate and/or electrically connect any terminal wire ends enclosed within.

The ear implant system may include a cochlear implant system, an auditory brainstem implant system, or a vestibular implant system. And embodiments of the present invention also include an ear implant system having an electrode arrangement according to any of the above.

### BRIEF DESCRIPTION OF THE DRAWINGS

FIG. 1 shows various anatomical structures in a human ear and some components of a typical cochlear implant system.

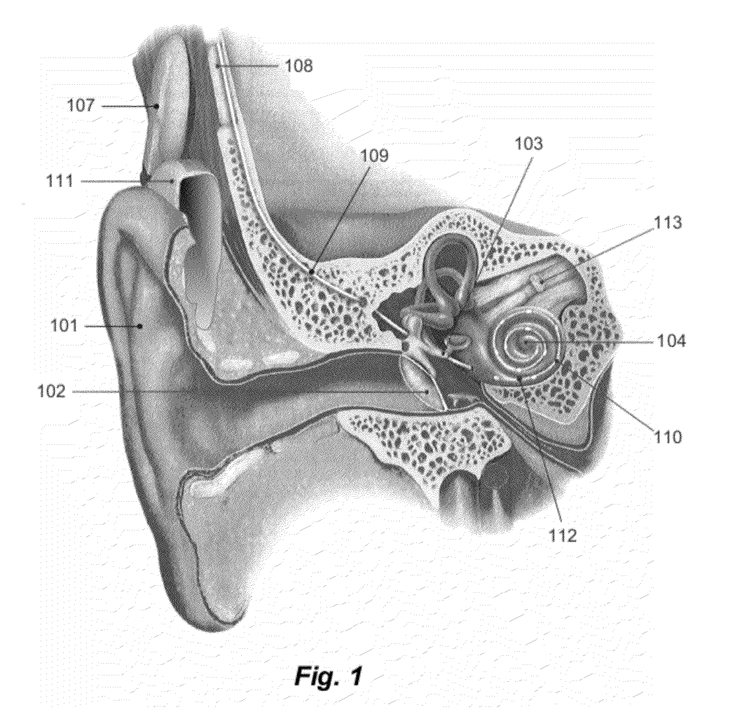

FIG. 2 shows an example of one specific embodiment of the present invention.

FIG. 3 shows details of an ABI electrode array according to one embodiment.

FIG. 4 shows details of a connector cap according to one embodiment.

FIG. 5 shows details of a cochlear implant electrode array according to one embodiment.

### DETAILED DESCRIPTION

Design engineers try to design the most atraumatic implant electrodes and insertion techniques so that new patients with residual hearing can have access to auditory prosthesis implants without degradation of their existing hearing. But many measurements cannot be performed from the implant itself except for potential field measurements which are a crude measurement dedicated to the whole nerve action potential and retransmission by telemetry which is not a real time measurement. More specifically, conventional telemetry measurements with the implant device are limited to extra cellular potential field generated by intra cellular action potential, or to recording potential distribution caused by current generated at one or more electrodes and recorded with the other electrodes. All signals are processed somewhat crudely by the implant electronics which does not allow for more precise or sophisticated measurements. Only a static measurement is available and no intra-operative real time measurement is possible as electrode insertion proceeds.

It would be most useful to provide an auditory prosthesis implant which could perform sophisticated dynamic measurements during implantation of the electrode in the target tissue. For example, during insertion of a cochlear implant electrode, such measurements could reveal forces, potential changes in the cochlear structure during insertion and placement of the electrode, allow recording of cochlear microphonics, chemical changes, concentration of some molecular species, pH changes etc. Having intra-operative access to such measurements would allow evaluation of the quality of the electrode insertion, preservation of tissue, amount of bleeding and injury inflicted on the tissue, perforation of basilar membrane and mixing of perilymph and endolymph, forces applied at different location, molecular generation of sub species, bleeding etc. These measurements would allow better understanding of the mechanism of insertion trauma with electrodes in real time.

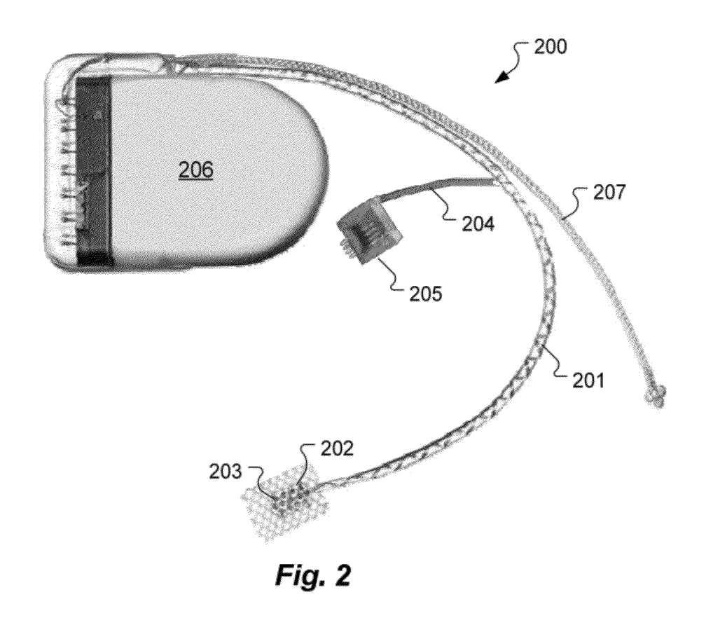

Embodiments of the present invention are directed to an improved implantable electrode arrangement for an auditory prosthesis system such as a cochlear implant or ABI system which allows for taking real-time intra-operative measurements as the implant electrode is inserted into or placed onto the target tissue. FIG. 2 shows an example of one specific embodiment of an ABI implant stimulator 206 which produces one or more electrical stimulation signals that are carried by electrode wires in an electrode lead 201 which connected at its base to the housing of the implant stimulator 206. At the apical end of the electrode lead 201 is an electrode array 202 with two types of contact surfaces 203. There is also a ground electrode that may be either part of the implant stimulator 206 or a separate ground electrode 207.

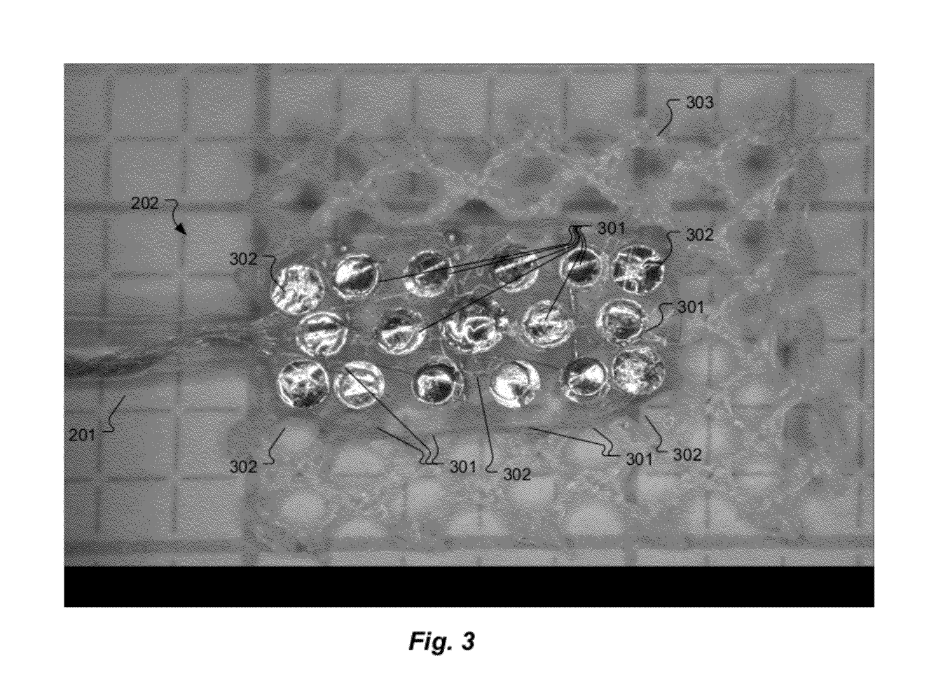

FIG. 3 shows the distal electrode array 202 in greater detail for an ABI implant system where there are conventional electrode contacts 301 that form a terminal end of each electrode wire for applying the electrical stimulation signals to the target neural tissue. Intra-operative sensors 302 can be at any specific position within the electrode array 202 and generate intra-operative sensing signals during surgical insertion of the electrode array 202 into the target tissue. In one specific embodiment, the intra-operative sensors 302 are on the edges of the electrode array 202. In a further embodiment, a further sensor 302 is in or close to the center of the electrode array 202. For example, the intra-operative sensors 302 could be separate electrodes for recording potential changes resulting from any loss of endocochlear potential (100 mV positive potential in the endolymph compared to perilymph). Or the intra-operative sensors 302 may specifically include a chemical or pH sensor. Or the intra-operative sensors 302 may be inductive sensors based on wire coils in the electrode array 202 and/or electrode lead 201 that sense changes in a magnetic field induced in near the target tissue. Besides electrical sensing contacts, the intra-operative sensors 302 also could be optical sensors connected to optical fibers. FIG. 3 also shows an array backing 303 of mesh fabric that provides a structure for post-operative tissue growth that helps permanently secure the electrode array 202 in position.

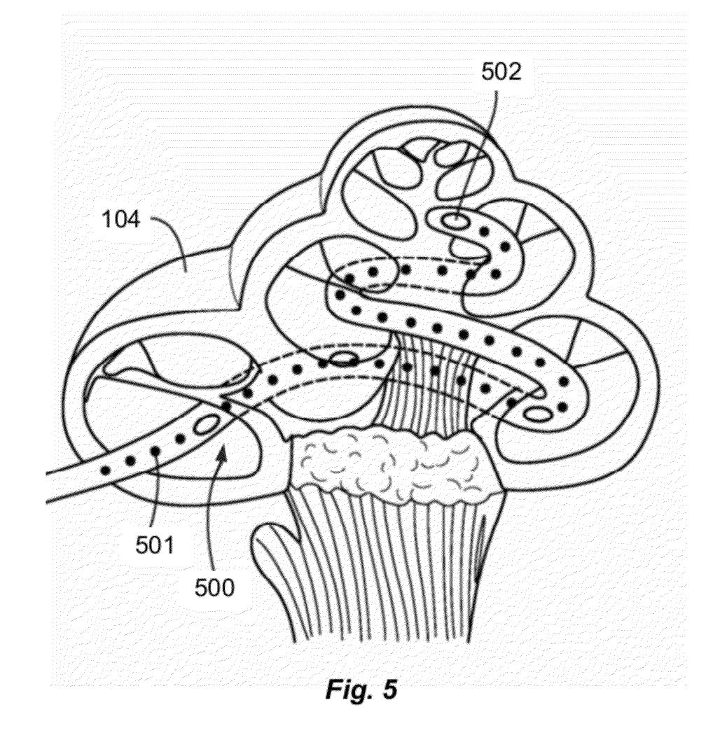

FIG. 5 shows the intracochlear electrode array 500 of a cochlear implant system, having conventional electrode contacts 501 that form a terminal end of each electrode wire for applying the electrical stimulation signals to the target neural tissue and intra-operative sensors 502. In a preferred embodiment the intra operative sensors 502 are equally spaced apart over the electrode array 500. The intra-operative sensors may be electrode contacts, preferred the same as the conventional electrode contacts 501, or fiber-optic terminal ends. In a further embodiment, the intra-operative sensors 502 are only on a part of the electrode array 500, e.g. the distal end of the electrode array 500.

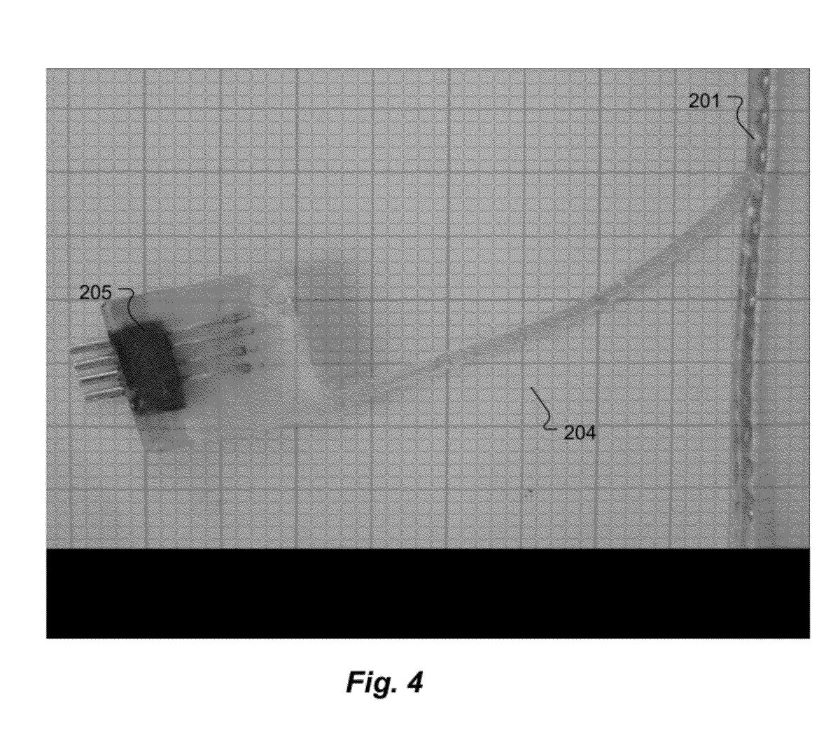

The electrode lead 201 also has a separate intra-operative electrode branch 204 that is connected to the intra-operative sensors 302. Intra-operative electrode branch 204 terminates in a sensor connector 205, in this case a 4 pin AXON connector for temporary connection of one or more external measurement arrangements during the surgical insertion of the electrode array 202. FIG. 4 shows the intra-operative electrode branch 204 and the sensor connector 205 in greater detail. The intra-operative electrode branch 204 and the sensor connector 205 are connected through another cable to sophisticated recording equipment located in the operating room for the intra-operative measurements. At the end of the surgery, the intra-operative electrode branch 204 is no longer needed and can be cut where it joins the electrode lead 201. Alternatively, the sensor connector 205 can be simply left in place and covered by a biocompatible cap made of silicone or other biocompatible material to protect it from surrounding body fluids. Such a cap may short circuit the connector pins or isolates them to each other. The sensor connector 205 pins may be of platinum-iridium alloy or other biocompatible material. In this way the covered sensor connector 205 could be placed in the mastoid cavity used again later in a subsequent surgery as needed.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable electrode arrangement for an ear implant system comprising:
a proximal electrode lead having a plurality of electrode wires for carrying one or more electrical stimulation signals;
a distal electrode array having:
i. a plurality of electrode contacts each forming a terminal end of an electrode wire for applying the electrical stimulation signals to target neural tissue, and
ii. one or more intra-operative sensors for generating insertion sensing signals during surgical insertion of the electrode array into the target tissue; and
an intra-operative electrode section having a sensor connector for providing a temporary connection of one or more external measurement arrangements to the one or more intra-operative sensors during the surgical insertion of the electrode array without being functional after the surgical insertion.

2. An electrode arrangement according to claim 1, wherein the insertion signals include dynamic real time sensing signals.

3. An electrode arrangement according to claim 1, wherein at least one of the intra-operative sensors is an optical sensor.

4. An electrode arrangement according to claim 1, wherein at least one of the intra-operative sensors is an inductive sensor.

5. An electrode arrangement according to claim 1, wherein at least one of the intra-operative sensors is a chemical sensor.

6. An electrode arrangement according to claim 1, wherein at least one of the intra-operative sensors is separate from the electrode contacts.

7. An electrode arrangement according to claim 1, further comprising:
a connector cap for covering the sensor connector following the surgical insertion of the electrode array into the target tissue.

8. An electrode arrangement according to claim 7, wherein the connector cap further is adapted to electrically isolate any terminal wire ends enclosed within.

9. An electrode arrangement according to claim 7, wherein the connector cap further is adapted to electrically connect together any terminal wire ends enclosed within.

10. An electrode arrangement according to claim 1, wherein the ear implant system includes a cochlear implant system.

11. An electrode arrangement according to claim 1, wherein the ear implant system includes an auditory brainstem implant system.

12. An electrode arrangement according to claim 1, wherein the ear implant system includes a vestibular implant system.

13. An ear implant system having an electrode arrangement according to any of claims 1-12.
